(19) United States
(12) Patent Application Publication
(10) Pub. No.: US 2025/0186246 A1
(43) Pub. Date: Jun. 12, 2025

(54) CONTOUR-FIT NASAL DILATOR

(71) Applicant: Breathewave Technologies Inc., Edgewater, NJ (US)

(72) Inventors: David J. Axelrad, Closter, NJ (US); Michael A. Linn, Ridgewood, NJ (US); Geoffrey P. Waite, Campton, NH (US); Christopher A. Beaudreau, Parker, CO (US)

(21) Appl. No.: 18/968,750

(22) Filed: Dec. 4, 2024

Related U.S. Application Data
(60) Provisional application No. 63/552,836, filed on Feb. 13, 2024.
(60) Provisional application No. 63/607,023, filed on Dec. 6, 2023.

(51) Int. Cl. A61F 5/56

(52) U.S. Cl. CPC A61F 5/56

(57) ABSTRACT

A nasal dilator is provided having one or more inserts that include one or more varied configurations that fit nasal passage anatomical configurations common to a group of potential users. The nasal dilator may include a circumferential force profile configured to vary at different points of contact along a circumference of the insert providing one or more dilation force magnitudes disposed at different locations around the circumference, wherein the one or more nasal inserts are shaped, sized, and configured based anatomical information of nasal passage surface features derived through an analysis of anatomical information from a group of potential users.

### CROSS-REFERENCE TO RELATED APPLICATION(S)

This application claims priority to, and the benefit of, co-pending U.S. Provisional Application 63/607,023, filed Dec. 6, 2023, and co-pending U.S. Provisional Application 63/552,836, filed Feb. 13, 2024, for all subject matter contained in all applications. The disclosures of said provisional applications are hereby incorporated by reference in their entirety.

### FIELD OF THE INVENTION

The present invention relates to nasal dilators suitable for increasing airflow through a user's nasal passages. In particular, the present invention relates to an anatomically shaped nasal dilator.

### BACKGROUND

Internal nasal dilators exist that have both solid-walled and latticed, or net, walled construction. These tend to be hollow conical frustums with a right-circular form, i.e., circularly symmetric. The therapeutic function of the upper ring and upper portion of the wall (typically, but not necessarily, smaller than the base ring) is to open and keep open the internal nasal valve while the base ring and lower portion of the wall open the external nasal valve. Both the two rings and the wall of the dilator also function to keep the dilator in place, sometimes with surface features such as ribs or bumps. These devices are typically fabricated of an elastic material creating an elastic spring force when distorted, such as a medical grade polymer like a silicone rubber or elastomer, such that when inserted into the nostril, they are somewhat accommodating of the nostril shape and excerpt a radial force to open and resist collapse of the nasal valves. In such existing devices, these forces can be determined based on the inherent elastic spring force of the simple hollow right circular, conical frustum form without considering the actual shape of the nasal passage, not the actual forces required for patency and comfort around the polar plane. Typically, such devices are offered in a limited number of size options, often four sizes, with both the ring diameters and the length of the device scaling appropriately.

However, this (technology, device, system, methodology, etc.) experiences some shortcomings. A problem with existing devices is that, since they do not accommodate the actual shape of the nostril, they exert an unequal dilation force around the circumference of the device when in use, resulting in suboptimal performance in both comfort and nasal patency efficacy.

### SUMMARY

There is a need for a nasal dilator that conforms more closely to the anatomical shape, location, and direction-dependent elastic modulus of the nose, thus allowing controlled distribution of dilation force around the circumference of the device such as to match the anatomical requirements to deliver a better optimized combination of patency and comfort. This contour fit design is achieved by an analysis of anatomical data and, from this analysis, creating a set of one or more dilator shapes matched to cohorts of user nostril shapes.

### BRIEF DESCRIPTION OF THE FIGURES

These and other characteristics of the present invention will be more fully understood by reference to the following detailed description in conjunction with the attached drawings, in which:

FIG. 1 depicts the structure of the human nose;

FIG. 2 depicts one embodiment of a nasal dilator system in accordance with embodiments of the present invention;

FIG. 3 depicts a force profile of a nasal insert of a nasal dilator system in accordance with embodiments of the present invention;

FIG. 4 depicts a configuration of a nasal dilator where the force profile is achieved by varying the structure and stiffness of various portions of the insert of a nasal dilator system in accordance with embodiments of the present invention;

FIG. 5 depicts a front view of another embodiment of an insert for a nasal dilator system in accordance with embodiments of the present invention;

FIG. 6 depicts a side view of the insert of FIG. 5;

FIG. 7 depicts the coronal plane of the insert of FIG. 5;

FIG. 8 depicts the sagittal plane of the insert of FIG. 6;

FIG. 9 depicts the connector bridge used with insert of FIG. 5 and FIG. 6;

FIG. 10 depicts the top view of the insert of FIG. 5 and FIG. 6;

FIG. 11 depicts the bottom view of the insert of FIG. 5 and FIG. 6;

FIG. 12 depicts example shapes for the top and base openings of the insert of FIG. 10 and FIG. 11;

FIG. 13 depicts different types of user noses for which the inserts of a nasal dilator can be configured;

FIG. 14 depicts different dilators for different nose types;

FIG. 15 is a CPAP pillow configuration; and

FIG. 16 depicts a nasal dilator with an adjustable common carrier mechanism connecting piece connecting the inserts.

### DETAILED DESCRIPTION

An illustrative embodiment of the present invention relates to a nasal dilator that conforms more closely to the anatomical shape of the nose, thus allowing controlled distribution of dilation force around the circumference of the device. This contour fit design is achieved by an analysis of anatomical data and, from this analysis, creating a set of one or more insert shapes matched to cohorts of user nostril shapes.

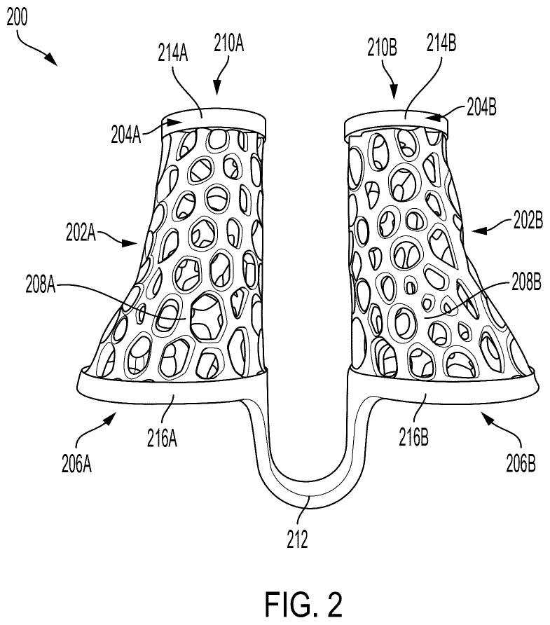

FIG. 2 through FIG. 16 wherein like parts are designated by like reference numerals throughout, illustrate an example embodiment or embodiments of a nasal dilation system, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

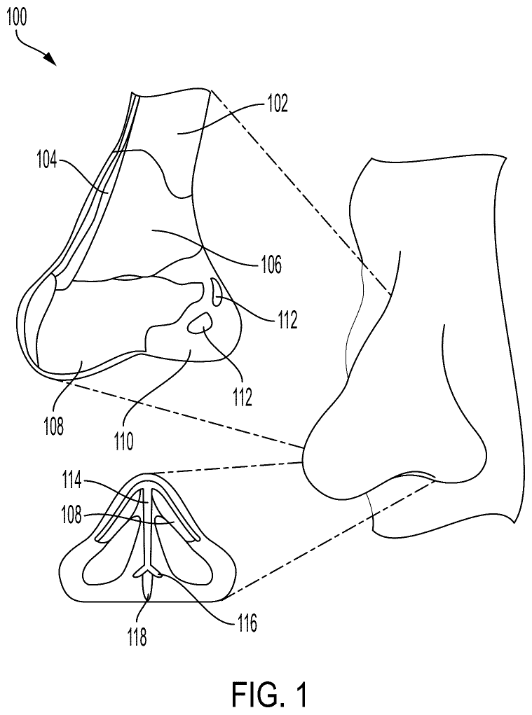

The human nose has a number of anatomical features that may affect the fit and operation of a nasal dilator. FIG. 1 shows the structure of a typical human nose 100. As can be seen here, the components that make up the structure of the nose include the nasal bone 102, septal cartilage 104, lateral nasal cartilage 106, greater alar cartilage (lateral crust) 108, alar fibrofatty tissue 110, minor alar cartilage 112, medial crus 114, footplate 116, and nasal septal cartilage 118. The human nose 100 has two valves: the internal and the external nasal valves. The internal nasal valve is level with the caudal border of the (upper) lateral nasal cartilage 106. This valve is a three-dimensional space bordered by the septum, the head of the lower turbinate, and the lateral cartilage and is not on a single plane. The internal nasal valve is the narrowest part of the entire airway. The external nasal valve includes all cartilaginous and soft tissue of the lateral nasal wall and that part of the septum, which is caudal to the internal nasal valve. A sub component of the external nasal valve can be considered to be the vestibular valve at the level of the vestibular rim. While some might describe the vestibular valve to be a discreet entity, we in include it in our definition of the external nasal valve.

It is well known to those skilled in the art that nose morphologies, including the two nasal valves, vary from person to person and that some traits can be common across different cohorts, which can be defined along different sets such as age, gender, racial or genetic lines, or simply morphological such as “bulbous”, “hook”, “pointed”, etc.

FIG. 2 depicts one example of the nasal dilation system 200 of the present invention. The nasal dilation system 200 comprises one or more nasal inserts 202A, 202B structurally adapted to align with a nasal passage surface anatomy common to a group of potential users where a group can be defined as narrowly as a cohort outlined above or as broadly as a single group for all persons. Each insert 202A, 202B comprises one or more varied configurations that fit nasal passage anatomical configurations common to at least one group of potential users and/or a circumferential force profile configured to vary at different points of contact along a circumference of the insert providing one or more dilation force magnitudes disposed at different locations around the circumference. The one or more nasal inserts 202A, 202B are shaped, sized, and configured based on anatomical information of nasal passage surface and structural features derived through an analysis of anatomical information from the group of potential users.

The nasal dilation system 200 of the present invention provides improvement of breathing by nasal dilation, which reduces constriction, achieved both by widening or opening and by holding open the nasal valve area of the nose 100. Constriction of the nasal valve can be continuous, e.g., in someone with a collapsed or narrowed nasal valve (common after trauma and in old age, but also congenitally) or rhythmic, caused by the soft tissue of the nasal valve being “sucked in” by the venturi effect of breathing in. Nasal valve constriction can also be temporarily increased by swelling in the nasal passages from various causes, including injury, inflammation, and infection. Of the many manifestations of constricted nasal valves, the two most (but not only) likely impacted by the invention are i) snoring and/or sleep disturbance, including sleep apnea, and ii) athletic performance.

Nasal valve constriction can lead a person to learn to more frequently mouth breathe instead of nose breathing.

Thus, the target users for the inventive device include:
People who, either consistently or intermittently, snore or suffer other breathing-related sleep disturbances, for whom the device may reduce snoring and breathing-related sleep disturbances.
People with sleep apnea for whom, while the device may not be sufficient to stop sleep apnea, it may help to reduce the severity and frequency of occurrences; the device is not a substitute for other sleep apnea treatments such as CPAP.
Athletes of all ability levels wishing to enhance their cardiovascular performance by improved breathing enabled by using the device in training or competition.
Athletes of all ability levels wishing to learn to nose breathe more often during exertion, using the device as a training aid.
People who wish to learn to nose breathe more often in any situation, using the device as a training aid.
People who already use internal nasal dilators but who find existing dilators to have limited performance or who suffer from discomfort or skin/tissue health issues as a result of such use.
People who use external nasal dilators who may find that this device works better for them without introducing the disadvantages of other internal nasal dilators.
People with temporary nasal constriction caused by injury, inflammation and infection, or other reasons who wish to improve breathing in any circumstance, awake or asleep.
In the device CPAP pillow form, people using CPAP machines make the pillow more comfortable and increase airflow for a given pressure setting on the machine.

In some instances, the nasal dilation system 200 present invention may also relieve nasal congestion.

In some embodiments, each insert 202A, 202B includes a top opening 204A, 204B and a base opening 206A, 206B connected by at least one support 208A, 208B extending between the top opening 204A, 204B and base opening 206A, 206B forming a passage 210A, 210B or cannula extending between the top 204A, 204B and base 206A, 206B openings.

In certain embodiments, the top openings 204A and 204B are defined by upper rings 214A and 214B, while the base openings are defined by base rings 216A and 216B. The top opening 204A, 204B and base opening 206A, 206B may have any number of shapes and sizes, including wherein the top opening 204A, 204B and base opening 206A, 206B may even have different sizes or shapes. In even further embodiments, the top opening 204A and/or base opening 206A of one insert 202A may have different sizes or shapes from the top opening 204B and/or base opening 206B of the other insert 202B.

In certain embodiments, the at least one support 208A, 208B may comprise a wall. In some such embodiments, the at least one support 208A, 208B may comprise a mesh or cage structure.

In certain embodiments, the nasal dilation system 200 is formed of one or more of metal, plastic, polymer, and rubber. In certain embodiments, the nasal dilator system 200 is formed of a pliant material. In certain embodiments, the one or more nasal inserts 202A, 202B are comprised of a biocompatible material, such as for example, silicone, polyurethane, polyethylene terephthalate (PET), and polylactic acid (PLA). Those of ordinary skill in the art will appreciate that other biocompatible materials can be utilized in accordance with the requirements and teachings of the present disclosure.

It is the combination of the top opening 204A, 204B, base opening 206A, 206B, and at least one support 208A, 208B that provides the one or more varied configurations. That is, the size, shape, and configuration of each of the top opening 204A, 204B, base opening 206A, 206B, and at least one support 208A, 208B combine to make the one or more varied configurations. These can include curves, indents, bulges, and other variations in the size, shape, and configurations of the top opening, base opening, and at least one support.

In FIG. 2, the top opening 204A, 204B and the base opening 206A, 206B have different sizes and shapes. The support 208A, 208B is a mesh wall extending from the top opening 204A, 204B to the base opening 206A, 206B. As can be seen here, the mesh wall also has a varying shape and configuration. Here the inserts 202A, 202B are further connected by a connector bridge 212.

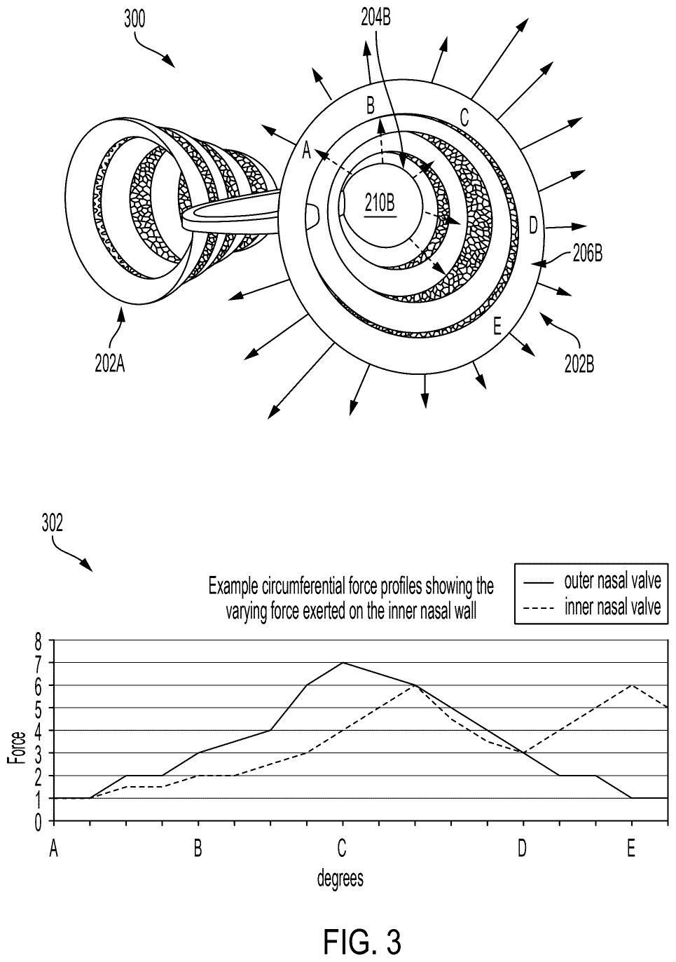

The dilation force is designed to differ at various points on the circumference, creating a circumferential force profile. The circumferential force profile can be designed to optimally trade off comfort and nasal patency (referring to an assessment of the openness or lack of obstruction of a nasal passage) by applying sufficient force at each point around the circumference to achieve good nasal patency without uncomfortable or excess force. The circumferential force profile can be designed to accommodate the variations in the underlying anatomical structure and soft tissue tone around the circumference, as well as the impact of dilation amount on nasal patency, which also varies around the circumference. The varying dilation force around the circumference may be achieved by one or more of a number of approaches, including geometric changes such as thickness, material type, material property modification, and mechanical structures such as lamination. An example of this can be seen in FIG. 3. In force depiction 300, the dilation force, indicated by the arrows, varies along the circumference as well as along the length of the insert 202B as indicated by the respective length of the arrow indicating the magnitude of the dilation force. Force plot 302 shows the magnitude of the dilation force around the circumference of the insert 202B.

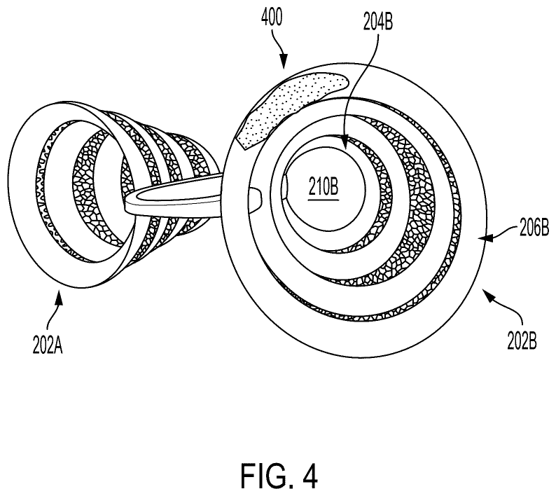

In one example, as seen in FIG. 4, the force profile of the structure of the base opening 206B varies around its circumference to exert a relatively larger force against the lateral processes of the greater alar/lateral crus cartilage 108, thus preventing inward collapse of the external valve by inward movement of the lateral crus cartilage 108; while also exerting a relatively lesser force against the minor alar cartilage 112 and the upper portions of the alar fibrofatty tissue 110, thus avoiding discomfort there. In this embodiment, the force profile is obtained by the use of a stiffer material 400 in the anterior portion of the structure of the base opening 206B combined with a thinner amount of material in the lateral and medial sections of the opening structure. Similarly, the structure of the top opening 204B may be profiled such as to exert relatively more force against the lower alar fibrofatty tissue 110 and relatively less force against the septal cartilage 104. Those skilled in the art will recognize that other circumferential force profiles would be appropriate for different classes of nasal anatomical shapes.

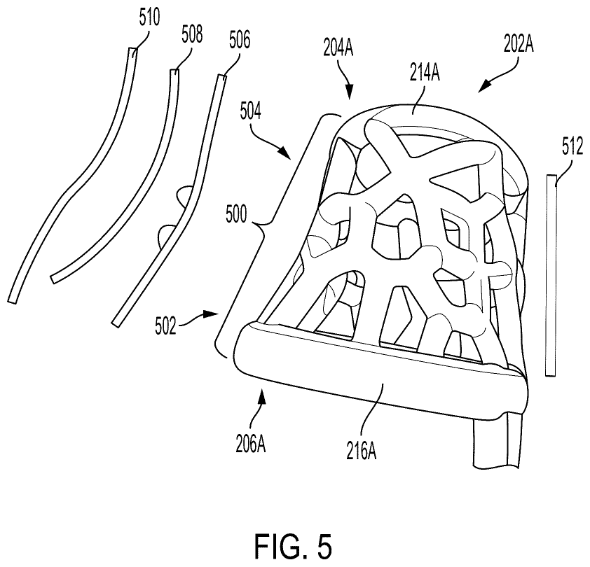

FIGS. 5-11 depict another embodiment of the nasal dilation system 200 of the present invention.

FIG. 5 depicts a front view of an insert 202A. In this embodiment, the lateral longitudinal profile 500 is designed to provide a proportionate opening force on the external nasal valve, i.e., the greater alar cartilaginous 108 structure and lesser/minor cartilaginous 112 structure, which in turn will exert a force on the inner nasal valve, i.e. lateral nasal cartilage structure 106 and septal cartilaginous 118 structure, rather than exerting most of the force on these superior cartilaginous structures directly by the dilator insert 202A. The insert profile 500 in the coronal plane is such that the tapering angle of the insert 202A is greater in the lower/caudal part 502 toward the base opening 206A than the higher/superior part 504 toward the top opening 204A. This can be achieved via a freeform design or a number of mathematical shapes, including lines interconnected by one or more vertices 506, an arc 508, a hyperbola, an “S” curve, a partial sine or hyperbolic sine shape 510, and others known to those skilled in the art. The medial profile 512 is designed to conform to the relatively straighter sagittal plane lateral aspect of the septal cartilage 104, 118 surface of the nostril 100 relative to the internal surface of the ala 108, 112.

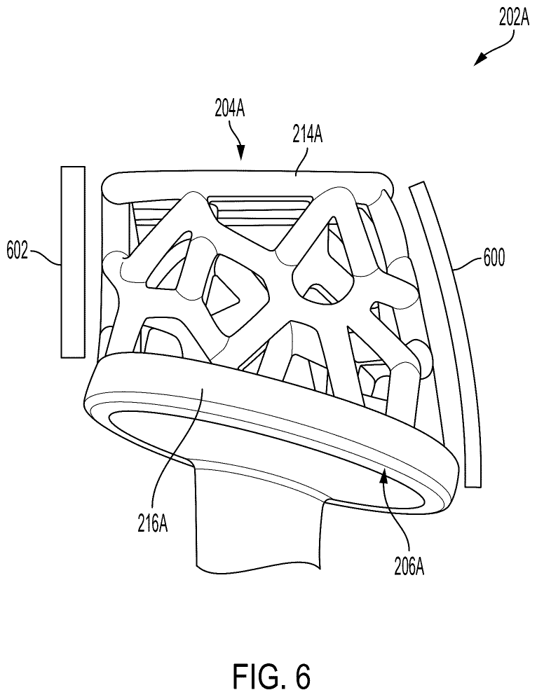

FIG. 6 depicts a side view of the insert 202A. Here, the graduated taper profile 600 is designed to conform to the anterior curve of nostril interior due to the shape of the nasal ridge formed by the profile of the septal cartilage 104 in the sagittal plane relative to the posterior surface of the insert 602 designed to engage with the posterior surface of the nostril. The profile 600 is also designed to be different than the lateral longitudinal profile 500 so as to conform to the medial surface of the septal cartilage 104, 118 of the nostril 100 relative to the internal surface of the ala 108, 112. This can be achieved via freeform design or via a number of mathematical shapes, including those depicted in FIG. 5, including lines interconnected by one or more vertices 506, an arc 508, a hyperbola, an “S” curve, a partial sine or hyperbolic sine shape 510, and others known to those skilled in the art.

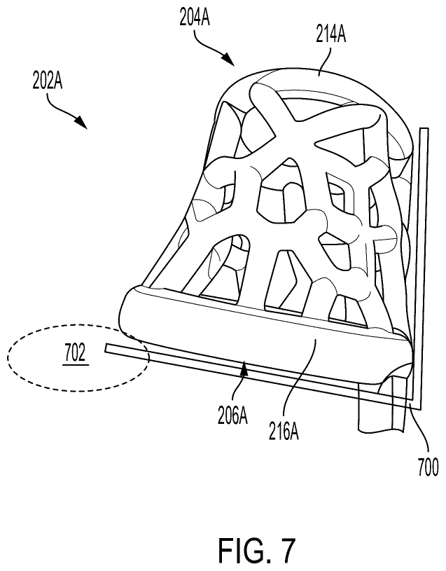

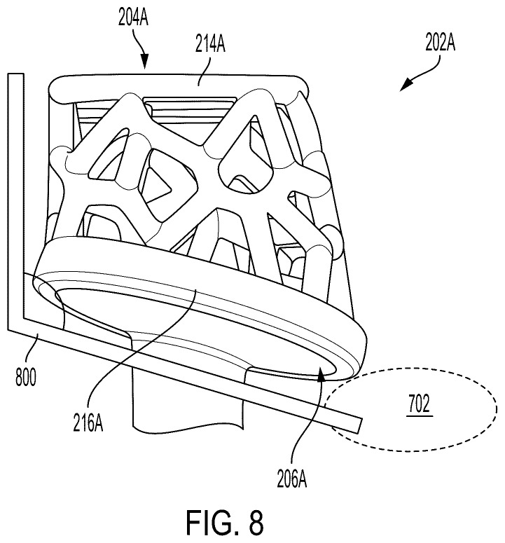

The insert 202A is designed to fit snugly inside a nostril with the base ring 216A defining the base opening 206A of the insert 202A positioned fully superior to the vestibular rim 512 of the nostril opening immersed, such that the vestibular rim 702 partially engages the lower/caudal surface of the base ring 216A of the base opening 206A of the insert 202A. The angles of the base ring 216A of the base opening 206A of the insert 202A to the medial septum edge of the insert 202A in both the coronal and sagittal planes are such as to engage the base ring 216A of the base opening 206A uniformly within the vestibular rim 702. Examples of this can be seen in FIG. 7 and FIG. 8. The angles of the base ring 216A of the base opening 206A may differ for different user groups, i.e., dilator shape selections. In one embodiment, the angle in the coronal plane 700 is an angle other than 90 degrees (+/−5 degrees) from the sagittal plane 800. In other embodiments, the angle in the coronal plane 700 is less than 85 degrees from the sagittal plane 800. In still further embodiments, the angle in the coronal plane 700 is less than 80 degrees from the sagittal plane 800. In alternate embodiments, the angle in the coronal plane 700 is more than 95 degrees from the sagittal plane 800. In further still alternates, the angle in the coronal plane 700 is more than 100 degrees from the sagittal plane 800. Similarly, in one embodiment, the angle in the sagittal plane 800 is an angle other than 90 degrees (+/−5 degrees) from the coronal plane 700. In other embodiments, the angle in the sagittal plane 800 is more than 95 degrees from the coronal plane 700. In still further embodiments, the angle in the sagittal plane 800 is more than 100 degrees from the coronal plane 700. Alternately, the angle in the sagittal plane 800 can be less than 85 degrees from the coronal plane 700. In still further alternates, the angle in the sagittal plane 800 is less than 80 degrees from the coronal plane 700.

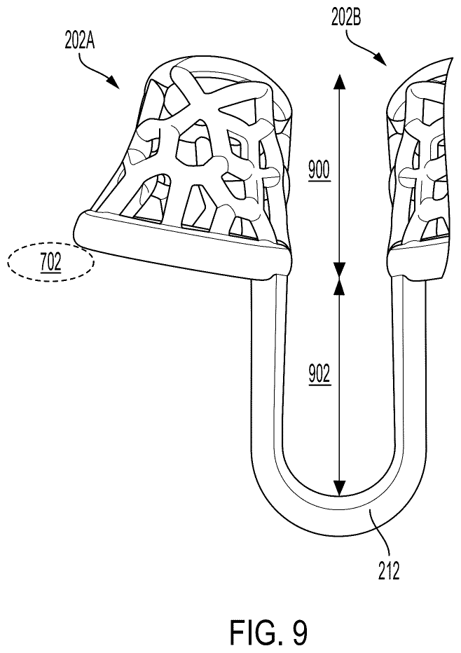

FIG. 9 depicts the connector bridge 212 used with this embodiment of the nasal dilation system 200. In certain embodiments, the length of the connecting bridge 902 is proportionately greater than the length 900 of the insert 202A, 202B to ensure that the inserts 202A, 202B are fully inserted into the nostril superior to the vestibular rim 702 such that the vestibular rim 702 partially engages the lower/caudal surface of the base ring 216A, 216B of the base opening 206A, 206B of the inserts 202A, 202B with no interference between the bridge 212 and the nasal columella and/or medial crus 114 of the nose 100. In one embodiment, the bridge length 902 is equal to or greater than the insert length 900. In another embodiment, the bridge length 902 is equal to or greater than 90% of the insert length 900. In still further embodiments, the bridge length 902 is equal to or greater than 75% of the insert length 900. In still further embodiments, the bridge length 902 is equal to or greater than 60% of the insert length 900.

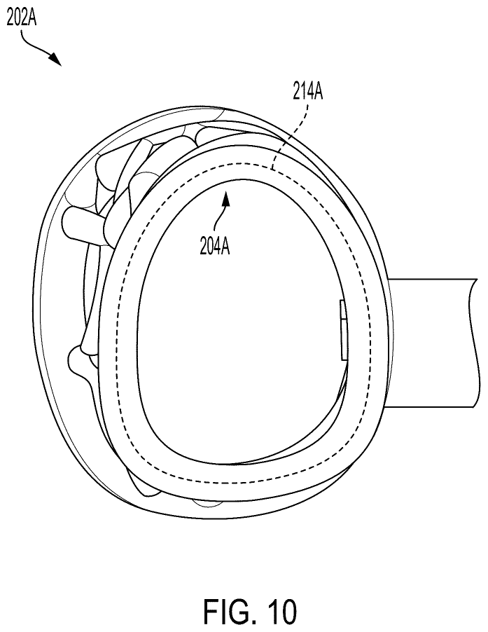

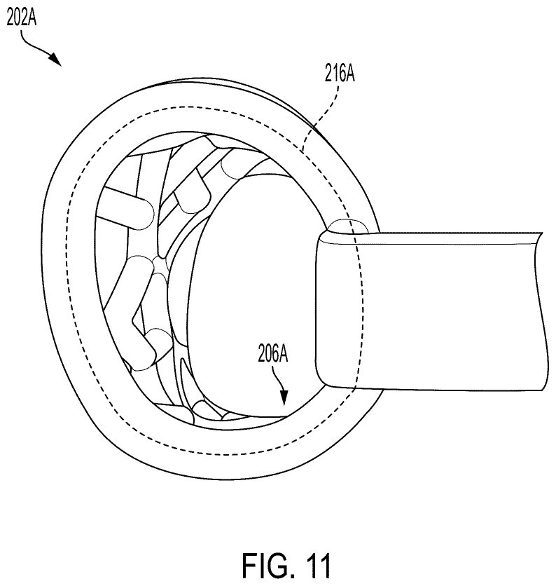

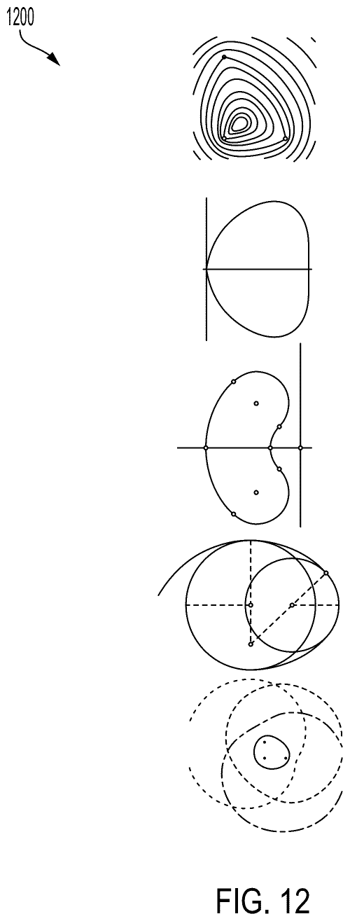

FIG. 10 depicts a top view of the insert 202A, while FIG. 11 depicts a bottom view of the insert 202A. In certain embodiments, the shape of the top ring 214A at the top opening 204A and base ring 216A at the base opening 206A are non-uniform, not approximately circular, or irregular in shape in order to better conform to the actual shape of a user's nostril. In some embodiments, one or both of the top ring 214A and base ring 216A have a generally ellipse shape. In other embodiments, one or both one or both of the top ring 214A and base ring 216A are formed in the shape of an oblong. In still other embodiments, one or both of the top ring 214A and base ring 216A are formed in the shape of an ovoid or egg. In further embodiments, one or both of the top ring 214A and base ring 216A have the shape of a 3-ellipse. In still further embodiments, one or both of the top ring 214A and base ring 216A have the shape of a n-ellipse, where n>3. In yet further embodiments, one or both of the top ring 214A and base ring 216A have the shape of a nephroid, fabiform, or bean curve. In alternate embodiments, one or both of the top ring 214A and base ring 216A are formed in a totally irregular, broadly convex shape or a totally irregular, broadly convex shape containing one or more concavities and other shapes known to those skilled in the art. Examples of some possible mathematical shapes 1200 can be seen in FIG. 12.

The relationship between anatomical nasal geometries and the optimum contour fit profile and circumferential force profiles is a complex one derived by an analysis of anatomical data and/or from empirical data derived from experimentation and/or collection of efficacy data from users of the device, and optionally by use of machine learning techniques. This relationship forms one of the aspects of the current invention.

In one embodiment, just one shape is deployed in different scaled sizes. For example, the nasal dilator could be provided in small, medium, and large sizes.

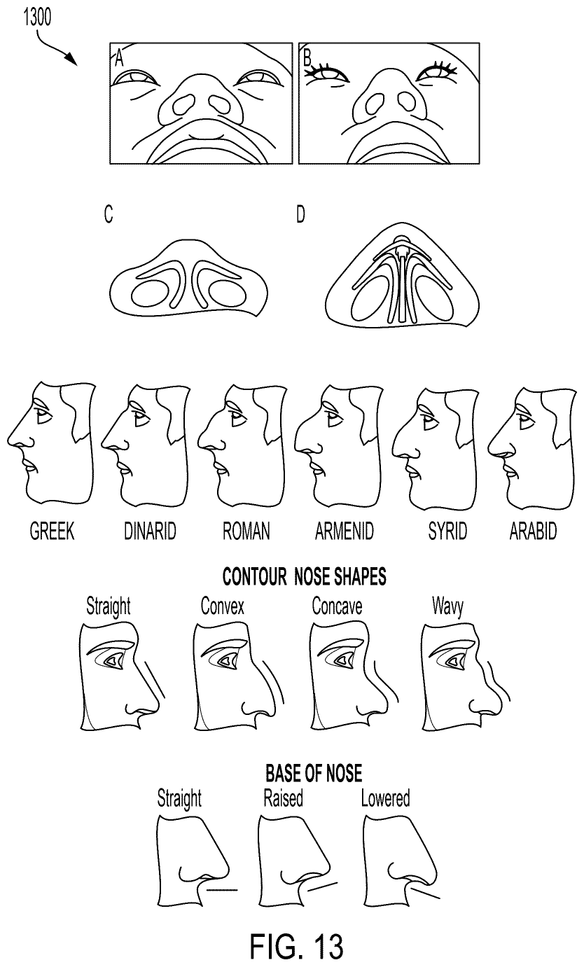

In another embodiment, a number of different shapes are provided to suit different nose and nostril anatomical shapes. Such variations might be described, for example, as thin, wide, high bridge, flared nostril, etc. Various examples of different nose configurations 1300 can be seen in FIG. 13.

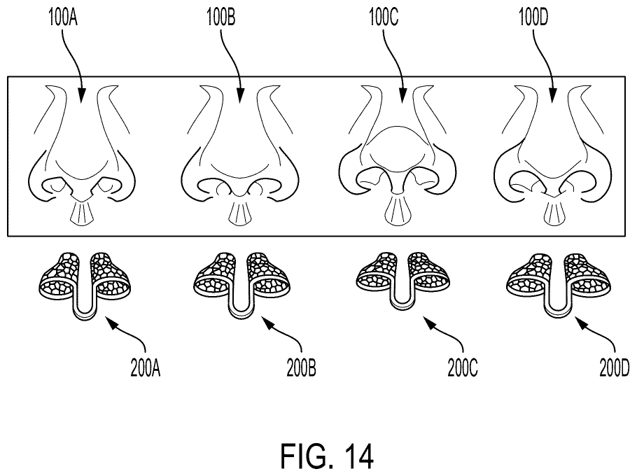

In another embodiment, a number of different shapes are provided to suit different nose and nostril anatomical shapes defined by racial or genetic origins. Such variations might be described, for example, as Asian, East African, West African, Northern European, Mediterranean, Indigenous Antipodean, etc. An example of this can be seen in FIG. 14. Where systems 200A, 200B, 200C, 200D are provided in different configurations for different shaped noses 100A, 100B, 100C, 100D.

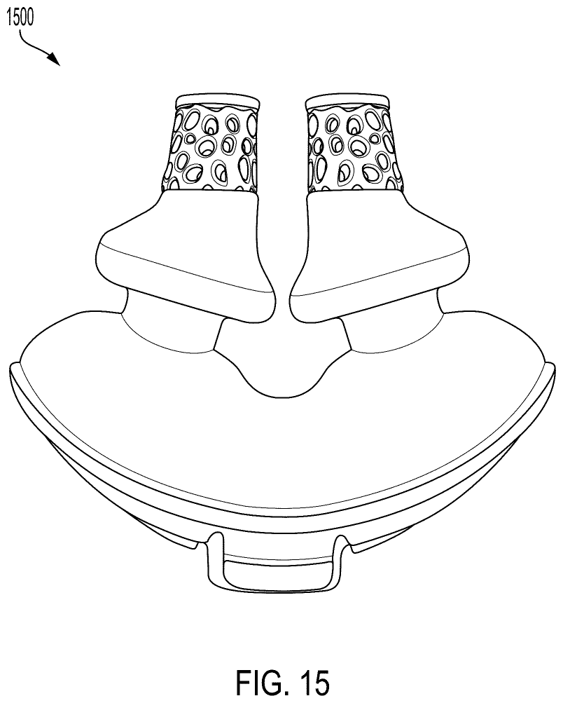

The invention is equally applicable to use in a CPAP “pillow” or system, where the same design principles used for the nasal dilator profiling are used to create a more comfortable and/or more securely located pillow insert effecting a higher nasal patency. An example of such a CPAP “pillow” 1500 can be seen in FIG. 15.

### Nostril Specific Feature

For some people with significant variance in nostril size or shape, for example, those with a deviated septum, having mirrored nasal inserts can present an obstacle to finding a configuration that provides both optimum comfort and optimum nasal patency in both nostrils, typically resulting in a compromise of both comfort and patency of the nasal passages.

As such, in certain embodiments, the nasal inserts 202A, 202B of the nasal dilation system 200 may be of different sizes (cross-sectional profile and/or heights), shapes, or configurations to accommodate such users and improve the trade-off between comfort and nasal patency, while at the same time avoiding the possibility of a single cone detaching and becoming lodged in the nose. In one such embodiment, the individual nose inserts 202A, 202B are selected and specified by the user, and a monolithic molded dilation system 200 having unequally sized and shaped inserts 202A, 202B are manufactured and supplied.

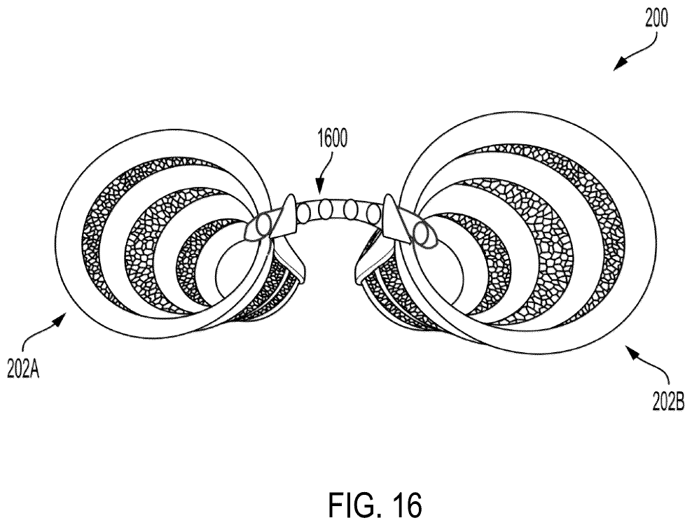

In another embodiment, the inserts 202A, 202B of the system 200 are connected to each other or to a common carrier mechanism in an irreversible assembly step. Examples of such assembly systems are well known to those in the art and include star-lock washers, pin clutches, and locking ratchets such as those used on zip ties, retail security labels, and the like. An example of this can be seen in FIG. 16. The common carrier 1600 can be a structure interconnecting two independent, stand-alone inserts or can be an integrated carrier forming a part of one or both of the inserts, for example, a spine of one side of the insert, or a bilateral core on which dilator inserts of different profiles or configurations are attached, in a similar way to size accommodations in earbuds.

### Fitting and Selection Feature

In certain embodiments, a method for helping the user identify the most suitable size and shape of the device may further be provided to help the user select an appropriate nasal dilator.

In one such embodiment, the user is presented with a range of templates and nose images and guided through a process of either positive or negative matching to their own nose to narrow down the choice of suitable device shapes and sizes. The templates and shapes can be presented either in hardcopy format, e.g., printed from a computer image, or via an electronic display device.

In a further embodiment, the user's own facial image can be superimposed on the templates or nose images to assist the user with matching. The superimposed images can contain visual cues, such as color-coding, to aid in matching.

In another embodiment, an image of the user's nose can be analyzed, and a shape and size match recommendation made by a computing system using image processing and computer vision.

In these embodiments, the images can consist of external images of the nose and face, for example, in plan and profile views. They can also consist of images of the nostrils internal profiles. In all cases, imaging and matching can be augmented by a calibration system such as printed fiducials, measurement rulings, or gratings.

In another embodiment, alternative imaging modalities can be used to obtain a measure of the user's nasal profile including, inter-alia, acoustic rhinometry, and x-ray imaging (including cone beam imaging). Those skilled in the art will appreciate that there are several known techniques to obtain such nasal profile data and that any of these might be deployed to assist in the fitting and selection of a contour-fit device appropriate to a specific user.

In another embodiment, nasal patency is measured by an instrument to provide the user with an objective measure of the most efficacious fit as they test different shape and size options. This instrument can be a rhinomanometer, measuring actual nasal flow rates, or can be a proxy for this, for example, using an acoustic breath signature and matching this to approximate flow rates. One known technique is the use of machine learning algorithms to make this estimation. In one embodiment of this approach, a personal device such as a mobile phone is used as both the data acquisition device, e.g., using the microphone, and the primary processing unit, optionally using an ancillary processing capability such as a cloud-based processor.

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may exist in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extent or degree of an action, characteristic, property, state, structure, item, or result, as would be appreciated by one of skill in the art. For example, an object that is “substantially” circular would mean that the object is either entirely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification, embodiments have been described in a way that enables a clear and concise specification to be written. Still, it is intended and will be appreciated that embodiments may be variously combined or separated without parting from the invention. The present invention is intended to be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A nasal dilation system, comprising:
one or more nasal inserts structurally adapted to align with a nasal passage surface anatomy common to a group of potential users, each insert comprising:
one or more varied configurations that fit nasal passage anatomical configurations common to a group of potential users; and/or
a circumferential force profile configured to vary at different points of contact along a circumference of the insert providing one or more dilation force magnitudes disposed at different locations around the circumference;
wherein the one or more nasal inserts are shaped, sized, and configured based upon anatomical information of nasal passage surface features derived through an analysis of anatomical information from the group of potential users.

2. The nasal dilation system of claim 1, wherein the one or more nasal inserts are formed of a pliant material.

3. The nasal dilation system of claim 2, wherein the one or more nasal inserts are formed of one or more of metal, plastic, polymer, and rubber.

4. The nasal dilation system of claim 1, wherein the one or more nasal inserts are comprised of a biocompatible material.

5. The nasal dilation system of claim 1, further comprising a longitudinal profile configured and arranged to provide an opening force on an external nasal valve of a user's nose upon placement therein, which exerts a force on an inner nasal valve of the user's nose.

6. The nasal dilation system of claim 1, wherein each nasal insert comprises:
a top opening;
a base opening; and
at least one support extending between the top opening and the base opening forming a passage extending between the top opening and the base opening.

7. The nasal dilation system of claim 6, wherein the at least one support comprises a wall.

8. The nasal dilation system of claim 7, wherein the wall comprises a mesh structure.

9. The nasal dilation system of claim 8, wherein the mesh comprises a varying shape and configuration.

10. The nasal dilation system of claim 6, further comprising an insert profile in a coronal plane comprising a tapering angle that is greater in a caudal part toward the base opening than a superior part toward the top opening.

11. The nasal dilation system of claim 10, wherein the insert profile comprises a freeform profile.

12. The nasal dilation system of claim 10, wherein the insert profile comprises one or more of: lines interconnected by one or more vertices, an arc, a hyperbola, an “S” curve, a partial sine or hyperbolic sine shape.

13. The nasal dilation system of claim 6, wherein the insert profile in a sagittal plane comprises a taper profile designed to conform to a curve of nasal ridge of a nose of a user.

14. The nasal dilation system of claim 6, wherein one or more of the top opening and bottom opening comprise a ring defining the opening.

15. The nasal dilation system of claim 6, wherein the top opening is sized, dimensioned, and configured for insertion within the nasal passages.

16. The nasal dilation system of claim 6, wherein the base opening is sized, dimensioned, and configured for insertion within the nasal insert in within the nasal passages.

17. The nasal dilation system of claim 16, wherein the base opening is sized, dimensioned, and configured to engage a vestibular rim of a nose of a user.

18. The nasal dilation system of claim 17, wherein the base opening is configured to have an angle in a coronal plane that is other than 90 degrees from a sagittal plane.

19. The nasal dilation system of claim 17, wherein the base opening is configured to have an angle in a coronal plane less than 85 degrees from a sagittal plane.

20. The nasal dilation system of claim 17, wherein the base opening is configured to have an angle in a coronal plane less than 80 degrees from a sagittal plane.

21. The nasal dilation system of claim 17, wherein the base opening is configured to have an angle in a coronal plane more than 95 degrees from a sagittal plane.

22. The nasal dilation system of claim 17, wherein the base opening is configured to have an angle in a coronal plane more than 100 degrees from a sagittal plane.

23. The nasal dilation system of claim 17, wherein the base opening is configured to have an angle in a sagittal plane that is other than 90 degrees from a coronal plane.

24. The nasal dilation system of claim 17, wherein the base opening is configured to have an angle in a sagittal plane more than 95 degrees from a coronal plane.

25. The nasal dilation system of claim 17, wherein the base opening is configured to have an angle in a sagittal plane more than 100 degrees from a coronal plane.

26. The nasal dilation system of claim 17, wherein the base opening is configured to have an angle in a sagittal plane less than 85 degrees from a coronal plane.

27. The nasal dilation system of claim 17, wherein the base opening is configured to have an angle in a sagittal plane less than 80 degrees from a coronal plane.

28. The nasal dilation system of claim 6, wherein one or more of the top opening and bottom opening comprises a non-uniform, not approximately circular, or irregular shape to better conform to a shape of a user's nose.

29. The nasal dilation system of claim 6, wherein one or more of the top opening and bottom opening comprises an ellipse shape.

30. The nasal dilation system of claim 6, wherein one or more of the top opening and bottom opening comprises an oblong shape.

31. The nasal dilation system of claim 6, wherein one or more of the top opening and bottom opening comprises an ovoid shape.

32. The nasal dilation system of claim 6, wherein one or more of the top opening and bottom opening comprises an n-ellipse shape, wherein n is greater than 2.

33. The nasal dilation system of claim 6, wherein one or more of the top opening and bottom opening comprises a nephroid, fabiform, or bean curve.

34. The nasal dilation system of claim 6, wherein one or more of the top opening and bottom opening comprises a totally irregular, broadly convex, shape.

35. The nasal dilation system of claim 34, further comprising one or more concavities.

36. The nasal dilation system of claim 1, comprising two nasal inserts.

37. The nasal dilation system of claim 36, further comprising a connector bridge connecting the two nasal inserts.

38. The nasal dilation system of claim 37, wherein the connecting bridge comprises a length that is equal to or greater than a length of the inserts.

39. The nasal dilation system of claim 37, wherein the connecting bridge comprises a length that is equal to or greater than 90% a length of the inserts.

40. The nasal dilation system of claim 37, wherein the connecting bridge comprises a length that is equal to or greater than 60% a length of the inserts.

41. The nasal dilation system of claim 37, further comprising a common carrier mechanism connecting the two nasal inserts having different profiles and/or configurations.

42. The nasal dilation system of claim 1, wherein the system is configured as part of a CPAP pillow.
